(19) United States
(12) Patent Application Publication
(10) Pub. No.: US 2026/0357181 A1
(43) Pub. Date: Nov. 12, 2026

(54) PERFUSION CELL CULTURE SYSTEMS AND METHODS

(71) Applicant: ALPHINITY, LLC, Carson City, NV (US)

(72) Inventor: Michael C. Gagne, Carson City, NV (US)

(73) Assignee: ALPHINITY, LLC, Carson City, NV (US)

(21) Appl. No.: 19/717,387

(22) Filed: Jun. 23, 2026

Related U.S. Application Data
(63) Continuation of application No. PCT/US2025/011729, filed on Jan. 15, 2025.
(60) Provisional application No. 63/621,461, filed on Jan. 16, 2024.

(51) Int. Cl. C12M 1/00; C12M 1/26

(52) U.S. Cl. CPC C12M 29/10; C12M 23/40; C12M 29/14; C12M 29/16; C12M 29/18; C12M 33/14

(57) ABSTRACT

A perfusion cell culture method and system are provided to filter cellular biomolecules in a perfusion operation. The method involves pumping the contents of a bioreactor or vessel containing cells into the inlet of a multi-outlet pump. The multi-outlet pump pumps the fluid containing the cells into a plurality of hollow fiber filters. The hollow fiber filters are coupled to a secondary pump such as a peristaltic pump to apply vacuum or negative pressure to the exterior or extra-capillary space located outside of the fibers of the hollow fiber filters. Waste and/or product passes from the interior, luminal space of the fibers into the exterior or extra-capillary space. This filtrate fluid containing the waste or product is then pumped out of the hollow fiber filters where it is captured. The fluid containing the cells, i.e., the retentate, is recirculated back to the bioreactor or vessel.

### RELATED APPLICATION

This Application is a continuation of International Patent Application Number PCT/US2025/011729, filed on Jan. 15, 2025, which itself claims priority to U.S. Provisional Patent Application No. 63/621,461 filed on Jan. 16, 2024. The above-noted patent applications are incorporated herein by reference. Priority is claimed pursuant to 35 U.S.C. § § 119, 120 and any other applicable statute.

### TECHNICAL FIELD

The technical field generally relates to perfusion cell culture systems and methods.

### BACKGROUND

Biopharmaceutical and biotechnology applications often involve the use of living cells which are grown in liquid growth media. The cells, which are typically mammalian cells, may generate one or more biomolecules that are the product of interest. In other applications, the cells themselves may be the desired end product. The cells are typically grown in vessels called bioreactors that hold the cells along with growth media that allow the division and growth of cells. Product yields are often a function of cell density so there is a need within the field to quickly grow and maintain cells at optimum densities to maximize product yields. Bioreactors may be run in batch, fed-batch, or semi-continuous modes. Continuous mode operations are desirable because high densities of cells may be maintained for long periods of time, thereby improving overall product yield. Perfusion cell culture refers to the culturing of cells with the use of one or more cell retention devices that retain cells within a bioreactor or fluid circuit incorporating the same while waste, products, or used media are continuously removed. Fresh media is typically added at the same rate at which use media is removed from the bioreactor. Perfusion cell culture has the advantage of being able to achieve high cell densities and high cell viabilities over a longer period of time, thereby allowing one to achieve high product yields.

One type of cell retention device used with perfusion cell culture includes hollow fiber filters. Hollow fiber filters are formed in a tubular cartridge or housing that contains many semipermeable hollow fibers arranged in a parallel array. The tubular housing includes inlet and outlet ports at either end of the housing that communicate with the luminal (intra-capillary) space inside the hollow fibers arranged in the housing. Another set of ports are arranged on the cartridge or housing and provide access to the extra-capillary space that lies outside the luminal interior space of the hollow fibers. In some modes, culture media for the cells is pumped through the intra-capillary space of the hollow fibers while the cells are contained in the extra-capillary space. This allows products and nutrients to diffuse across the walls of the hollow fibers. For example, in one embodiment, cells are present in the extra-capillary space while media is pumped through the luminal space of the hollow fibers. The cells may attach and adhere to the outer surfaces of the fibers. Nutrients carried by this media may diffuse across the hollow fiber walls into the extra-capillary space to aid in cell growth and viability. Metabolic products of the cells (e.g., product or waste) may be transported from the extra-capillary space and into the luminal space of the hollow fibers where the products and may be captured and/or disposed of. In other modes of operation, however, the cells may be located in the intra-capillary space inside the fibers while the extra-capillary space is free from cells. The cells thus remain in the retentate contained within the fibers while a filtrate or permeate is formed in the extra-capillary space.

### SUMMARY

In one embodiment, a bioreactor system is provided that uses a single multi-outlet pump. A bioreactor or vessel includes an outlet that is connected to an inlet of a single multi-outlet pump. Tubing or conduit may connect the outlet of the bioreactor or vessel to the inlet of the single multi-outlet pump. The outlets of the multi-outlet pump are fluidically connected via tubing or conduit to a plurality of separate hollow fiber filters. Preferably, each outlet of the multi-outlet pump is coupled to a separate hollow fiber filter. Each hollow fiber filter includes a housing that holds a plurality of hollow fibers held in a parallel array and potted against respective ends of the housing. An inlet and an outlet are provided on the filter housing which communicates with the interior, luminal space inside the hollow fibers contained with the hollow fiber filter. Fluid flows into the intra-capillary space of the hollow fibers via the inlet and leaves the hollow fiber filter via the outlet. A first port and a second port are disposed on the housing of each hollow fiber filter and communicates with the exterior or extra-capillary space located outside of the fibers. The filtrate, which is contained in the extra-capillary space, may then be removed from the hollow fiber filter with a peristaltic pump. Tubing or conduit connects each of the outlets of the multi-outlet pump to respective inlets of the hollow fiber filters. In this configuration, cells enter the interior, intra-capillary space inside the hollow fibers.

The outlets of the respective hollow fiber filters are connected to tubing or conduit that leads back to the bioreactor or vessel. In one embodiment, a manifold is interposed in the return path between the plurality of hollow fiber filters and the bioreactor or vessel. For example, the manifold may include a plurality of respective inlets for receiving fluid and cells leaving the hollow fiber filters. The manifold includes a single outlet that is fluidically connected to the bioreactor or vessel via a conduit or tubing. In other embodiments, the manifold also receives additives which are combined with the retentate and fed back to the bioreactor or vessel.

One or more of the ports of each of the plurality of hollow fiber filters is connected via conduit or tubing to a pump that pulls a vacuum or negative pressure within the exterior or extra-capillary space located outside of the fibers. For example, the pump may include a peristaltic pump. This pump aids in pulling cell-free fluid that contains metabolic waste, and/or products. In one embodiment, each of the hollow fiber filters includes the same pore or membrane size. The hollow fiber filters may be used to extract, for example, the desired product that is generated by the cells. Alternatively, the hollow fiber filters may be used to extract, for example, waste products that are to be discarded. In another embodiment, one or more of the hollow fiber filters is used to generate an enriched fraction fluid containing product(s) while one or more of the hollow fiber filters is used generate an enriched fraction of fluid containing waste product(s). This may be accomplished by using hollow fiber filters having different molecular weight cutoffs. This last embodiment allows the simultaneous filtration of different metabolic products from the cells. For example, waste removal may be performed at the same time as product removal. Waste removed via one or more hollow fiber filters may be directed to a vessel or container while the outlets from the one or more hollow fiber filters removing product may be directed to a separate vessel or container.

There are several advantages to using a single multi-outlet pump for bioreactor perfusion operations. A first benefit is that a reduced footprint is required for the perfusion operation. Space for biopharmaceutical processes and operations is often limited and very expensive and reducing this footprint enables perfusion operations to be performed in a smaller, compact space. Instead of having multiple pumps and associated electronics and fluidic connections, a single pump is used. Another benefit is that the flow rates to each of the hollow fiber filters is controlled by a single pump. Equal flow is provided to the hollow fiber filters by a single pump. If there were separate pumps associated with each hollow fiber filter uniform flow rates to each hollow fiber filter might not be possible. Moreover, complicated and expensive control electronics would be required for each pump to provide a consistent flow rate to the hollow fiber filters. Moreover, there is no use of a manifold prior to the hollow fiber filters which may introduce inevitable flow perturbations to the hollow fiber filters. Another advantage is that the single multi-outlet pump contemplated herein operates without the pulse flow of other pumps which can adversely affect cell growth conditions.

In one embodiment, a method is provided to filter one or more cellular metabolites or products in a perfusion operation. The method involves pumping the contents of a bioreactor or vessel containing cells into a multi-outlet pump. The multi-outlet pump pumps the fluid containing the cells into a plurality of hollow fiber filters. The hollow fiber filters are coupled to a secondary pump such as a peristaltic pump to apply vacuum or negative pressure to the exterior or extra-capillary space located outside of the fibers of the hollow fiber filters. Waste and/or product passes from the interior, luminal space of the fibers into the exterior or extra-capillary space located outside of the fibers of the hollow fiber filters. This fluid containing the waste and/or product is then pumped out of the hollow fiber filters where it is captured in storage (e.g., waste storage or product storage). The fluid containing the cells is recirculated back to the bioreactor or vessel. Additional fluid (e.g., reagents, buffer, or the like) is added to the bioreactor or vessel to make-up for filtrate extracted from the hollow fiber filters.

The cell-laden fluid that passes through the luminal space of the fibers also contains biomolecules. The biomolecules may include metabolites, waste products, proteins or protein fragments, nucleic acids, cell secretions, and the like. Some of the biomolecules and other molecules contained within the cell-laden fluid are able to pass through the wall of the hollow fibers. Depending on the or molecular cutoff size that is used for the hollow fibers, molecules below the cutoff size are able to traverse the hollow fiber walls and enter the extra-capillary space where they are removed from the hollow fiber filter. Conversely, cells or molecules above the molecular weight cutoff of the hollow fibers are unable to pass through the wall of the hollow fibers and are thus retained in the luminal space inside the hollow fibers. These cells and molecules are unable to access the extra-capillary space and exit the hollow fiber filter via the outlet at the end where the cell-laden fluid is ultimately recirculated back to the bioreactor or vessel.

In some embodiments, different hollow fiber filters having different molecular cutoffs used in the respective hollow fiber filters are used. This may be used to generate different fractions of fluid (e.g., enriched product fluid and/or enriched waste fluid). This allows for simultaneous extraction of different product and/or waste fractions.

The number of hollow fiber filters may vary depending on the application. For example, there may be as few as two hollow fiber filters. Other embodiments may use larger numbers of hollow fiber filters (e.g., 3, 4, 5, 6, 7, 8, etc.). In some embodiments, the number of outlets of the multi-outlet pump equals the number of hollow fiber filters. Note that the one or more of the outlets of the multi-outlet pump, in some embodiments, may be closed. For example, an eight (8) outlet pump may pump cell-containing fluid to four (4) hollow fiber filters. In this embodiment, four (4) outlets of the pump are closed (e.g., with a cap, cover, or valve).

In another embodiment, the cell-laden fluid that passes through the hollow fiber filters is combined or mixed with various additives. These additives are provided in a fluid that is combined or mixed with the cell-laden return streams. The combination or mixing may take place in manifold that combines the fluids prior to return to the bioreactor or vessel. The additives may include reagents, cell growth media or supplements, base, acids, buffers, anti-foam agents, growth factors, and the like. The different additives may be contained in different containers, vessels, or bags that are individually pumped into the manifold prior to return to the bioreactor or vessel.

In one embodiment, the multi-outlet pump, hollow fiber filters, product/waste pump (e.g., peristaltic pump), manifold, and optional additives may be located on a common skid or cart that can be easily co-located with the bioreactor or vessel. The skid or cart further includes a computer or controller for controlling the multi-outlet pump, peristaltic pump, and pumps associated with optional additive containers, vessels, or bags.

In one embodiment, a perfusion cell culture method includes fluidically coupling cell-laden fluid from a bioreactor or vessel into an inlet of a multi-outlet pump and pumping the cell-laden fluid out a plurality of outlets of the multi-outlet pump, the plurality of outlets are fluidically connected via tubing or conduits to separate hollow fiber filters. A filtrate solution is extracted from the hollow fiber filters using one or more pumps fluidically coupled to an extra-capillary space within the hollow fiber filters. The retentate containing the cells from the hollow fiber filters is returned back to the bioreactor or vessel.

In another embodiment, a perfusion cell culture system includes a bioreactor or vessel. The system includes a multi-outlet pump having an inlet fluidically connected to the bioreactor or vessel and a plurality of outlets. A plurality of hollow fiber filters are fluidically coupled at respective inlets to the plurality of outlets of the multi-outlet pump, wherein the respective inlets of the hollow fiber filters communicate with an intra-capillary space of a plurality of hollow fibers contained within respective hollow fiber filters, the hollow fiber filters further including respective outlets that communicate with the intra-capillary space of a plurality of hollow fibers contained within respective hollow fiber filters. The hollow fiber filters include one or more ports that communicate with an extra-capillary space surrounding the hollow fibers of the hollow fiber filters. A manifold is fluidically coupled to the bioreactor or vessel and the respective outlets of the plurality of hollow fiber filters. One or more pumps (e.g., peristaltic pumps) are fluidically coupled to the respective one or more ports of each of the plurality of hollow fiber filters to aid in filtrate removal.

In another embodiment, a perfusion cell culture system includes a bioreactor or vessel and a multi-outlet pump having an inlet fluidically connected to the bioreactor or vessel and a plurality of outlets. The system further includes a plurality of hollow fiber filters each having a plurality of hollow fibers therein and defining an intra-capillary space inside the plurality of hollow fibers and an extra-capillary space outside the plurality of hollow fibers, the plurality of hollow fiber filters fluidically coupled at respective inlet ports to the plurality of outlets of the multi-outlet pump, wherein the respective inlet ports of the hollow fiber filters communicate with the extra-capillary space of a plurality of hollow fiber filters, the hollow fiber filters further including respective outlet ports that communicate with the extra-capillary space of the plurality of hollow fiber filters, and an inlet and outlet that communicate with the intra-capillary space of the hollow fibers in the plurality of hollow fiber filters. A manifold is fluidically coupled to the bioreactor or vessel and the respective outlet ports of the plurality of hollow fiber filters. A second pump is fluidically coupled to the respective outlets of the plurality of hollow fiber filters that communicate with the intra-capillary space of the hollow fibers in the plurality of hollow fiber filters.

### BRIEF DESCRIPTION OF THE DRAWINGS

FIG. 1 is a perspective view of a bioreactor or vessel that is fluidically connected to a multi-outlet pump. The multi-outlet pump receives cell-laden fluid from the bioreactor or vessel and pumps the same out a plurality of outlets (three outlets illustrated in this specific embodiment). Tubing or conduit carries the fluid from each outlet to separate hollow fiber filters. The cell-laden fluid passes through the hollow fibers within the respective hollow fiber filters and exits the hollow fiber filter at an opposing end where the respective flows are combined in a manifold and the resultant fluid is returned to the bioreactor or vessel. A port on each of the hollow fiber filters that communicates with the extra-capillary space within each hollow fiber filter is connected to tubing or conduit that is connected to a peristaltic pump. The peristaltic pump applies a slight vacuum or negative pressure to aid in pulling fluid from the extra-capillary space of the hollow fiber filters. Another port of the respective hollow fiber filters may be used to introduce fluid into the extra-capillary space.

FIG. 2 is perspective view of a bioreactor or vessel that is fluidically connected to a multi-outlet pump according to another embodiment. In this embodiment, one or more additives are provided that are combined with the cell-laden retentate. The additives are illustrated as contained in flexible bags. Each bag is associated with a pump located at the bottom thereof that is used to pump the contents of each bag to a manifold that combines or mixes the additive(s) with the cell-laden retentate which then gets returned to the bioreactor or vessel. Also illustrated is a tote that holds container(s) for receiving the filtrate from the hollow fiber filters.

FIG. 3 is another perspective view of the perfusion cell culture system illustrated in FIG. 2.

FIG. 4 is a top view of the perfusion cell culture system illustrated in FIG. 2.

FIG. 5A is a cross-sectional view of a single hollow fiber filter. Cells and media are illustrated as flowing into the hollow fibers (intra-capillary space). Filtrate is removed via port(s) from the extra-capillary space that surrounds the fibers.

FIG. 5B is a cross-sectional view of a single hollow fiber showing the intra-capillary space and the extra-capillary space. In addition, the flow across the semi-permeable hollow fiber is illustrated (arrow originating from intra-capillary space and exiting to the extra-capillary space).

FIG. 6 is a cross-sectional view of the manifold used to combine the retentate flows from the hollow fiber filters as well as the optional additives from the flexible bags illustrated in FIGS. 2-4.

### DETAILED DESCRIPTION OF THE ILLUSTRATED EMBODIMENTS

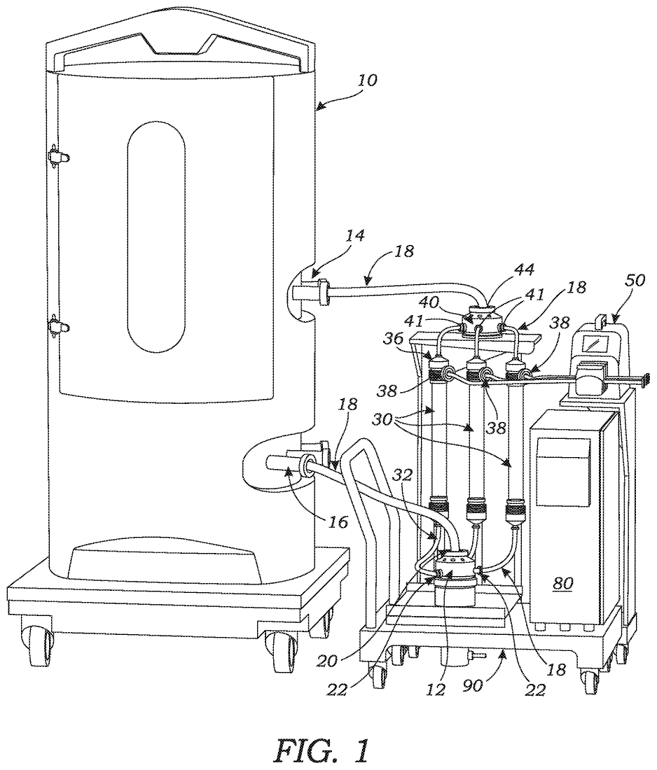

FIG. 1 illustrates a perspective view of a bioreactor or vessel 10 that is fluidically connected to the multi-outlet pump 12. The bioreactor or vessel 10 illustrated in FIG. 1 may be a bag or other container that is contained within a holder as illustrated. The bioreactor or vessel 10 may have a volume that varies depending on the application. Small volumes of less than a liter are possible. In still other applications, the bioreactor or vessel 10 may have volumes on the order of tens or hundreds of liters. The bioreactor or vessel 10 has at least one inlet 14 that receives the recirculated cell-laden retentate as explained herein. Additional inlets 14 may also be included in the bioreactor or vessel 10 to add cell culture media or the like. The bioreactor or vessel 10 contains the cell culture that includes live cells contained in media. The cells are typically mammalian cells but the invention is not limited to any particular cell type. The bioreactor or vessel 10 includes an outlet 16 that is connected to tubing or conduit 18 that is coupled to an inlet 20 of the multi-outlet pump 12. Additional outlets 16 may be provided in the bioreactor or vessel 10 that may be used, for example, for cell harvest. The multi-outlet pump 12 receives cell-laden fluid from the bioreactor or vessel 10 and pumps the same out a plurality of outlets 22 (three outlets 22 are illustrated in this specific embodiment). Tubing or conduit 18 carries the fluid from each outlet 22 to separate hollow fiber filters 30.

The multi-outlet pump 12 may include a multi-outlet pump 12 of the type described in U.S. Patent Application Publication No. 2023/0358223 (ALPHINITY USA, INC.), which is incorporated by reference herein. This multi-outlet pump 12 operates as a diaphragm or membrane pump. A diaphragm pump or membrane pump operates as positive displacement pump that uses moving diaphragm(s) in combination with check valves to pump fluid. In this multi-outlet pump 12 the drive shaft of a motor or drive unit is secured to a pump head and is used to drive a nutating disk or wobble plate to actuate the diaphragm membranes to drive fluid through the multi-outlet pump 12. For example, the nutating disk or wobble plate interfaces with a lower actuator disk or ring that sequentially actuates each of the diaphragms upon the wobbling motion of the nutating disk or wobble plate. Alternatively, servo motors or electronic/magnetic actuators may be used to sequentially actuate the diaphragm membrane(s) to achieve a similar pumping action. The multi-outlet pump 12 includes an inlet 20 at the top or upper region that receives the incoming fluid from the bioreactor or vessel 10. The multi-outlet pump 12 includes a plurality of outlets 22. The number of outlets 22 may vary (e.g., 2-10 or more outlets). In some embodiments, if all of the outlets 22 are not used, they may be closed via a cap, cover, or with the aid of a valve.

The multi-outlet pump 12 has an outer chamber that is fluidically connected to the inlet 20. A plurality of lower chambers are disposed in the multi-outlet pump 12 beneath the outer chamber and fluidically connected to the outer chamber by respective check valves interposed between the outer chamber and the plurality of lower chambers. A central chamber is disposed in the multi-outlet pump 12 that is fluidically connected to the plurality of lower chambers with respective check valves interposed between the central chamber and the plurality of lower chambers. The multi-outlet pump 12 has a plurality of outlets 22 that are fluidically coupled to the central chamber. Optional check valves may be provided in or prior to the outlets 22 to prevent the backflow of fluid into the central chamber. A moveable diaphragm is disposed in each of the plurality of lower chambers. The moveable diaphragms interface with a respective actuating element driven by a wobble or nutating plate that is operatively coupled to a motor or drive unit. Rotation of the shaft of the motor or drive unit thereby causes (with the aid of the wobble or nutating plate) each of the moveable diaphragms to move in opposing direction (e.g., up and down). This movement pumps fluid through the multi-outlet pump 12. The flow of the cell-laden fluid through the multi-outlet pump 12 initiates in the inlet 20 then into the outer chamber where the fluid then enters the plurality of lower chambers via respective check valves. From the lower chambers, fluid then is forced through check valves into the central chamber and out the outlets 22.

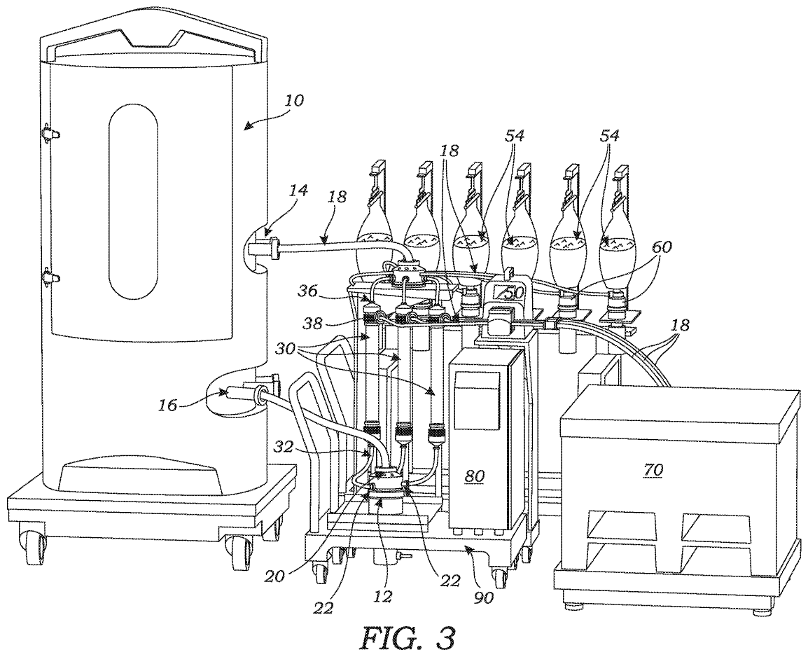

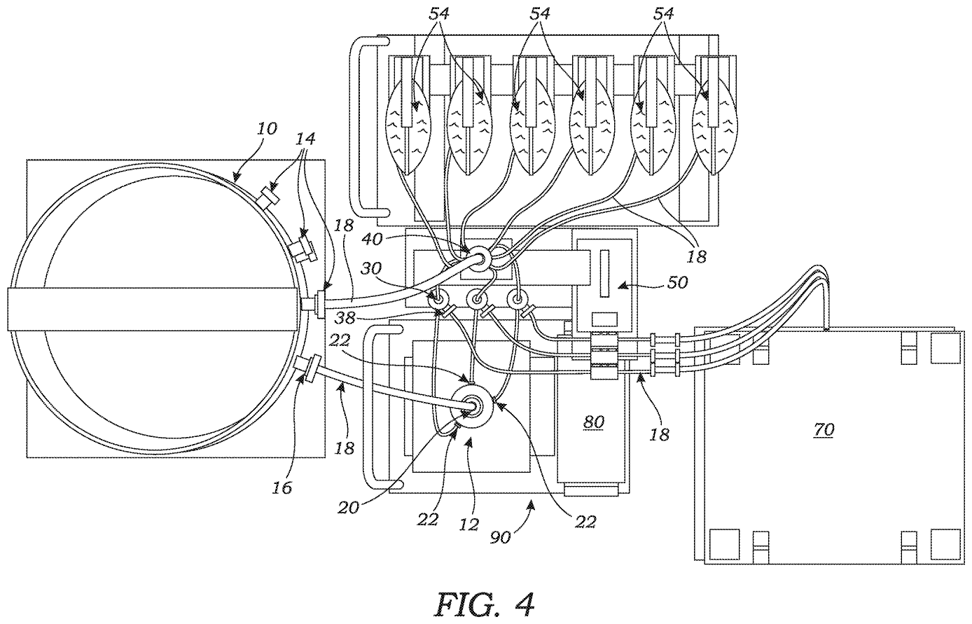

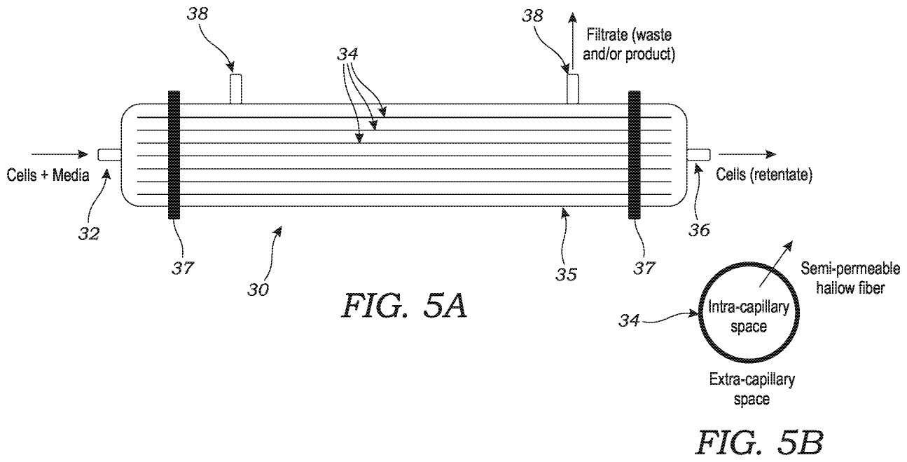

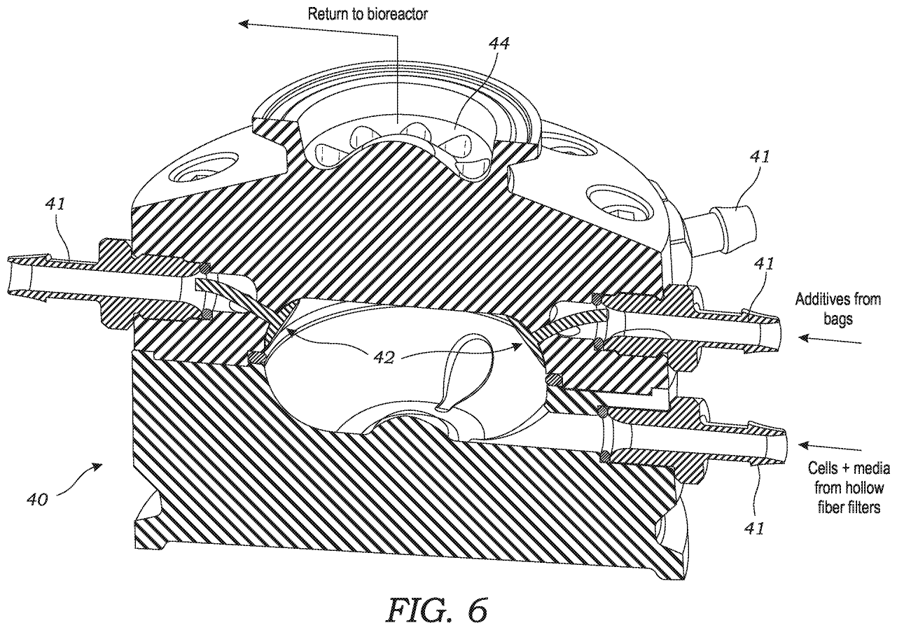

The cell-laden fluid passes from the multi-outlet pump 12 and into the hollow fiber filters 30. FIG. 5A illustrates an example of a hollow fiber filter 30 that is used with the systems and methods described herein. Specifically, the cell-laden fluid enters an inlet 32 of the hollow fiber filter 30 and proceeds into the luminal (i.e., intra-capillary) space of the hollow fibers 34 and exits the hollow fiber filter 30 at an opposing end via an outlet 36. The hollow fibers 34 are potted against respective ends 37 of the housing 35. Tubing or conduit 18 connect the outlet 36 of each hollow fiber filter 30 to a manifold 40 (FIGS. 1-4). The manifold 40 combines the respective flows from the outlets 36 of each hollow fiber filter 30 before being returned to the bioreactor or vessel 10 via an outlet 44 (FIG. 6) of the manifold 40. One or more ports 38 on each of the hollow fiber filters 30 fluidically communicates with the extra-capillary space within each hollow fiber filter 30. The port(s) 38 is/are connected to tubing or conduit 18 that is connected to a pump 50, which in one preferred embodiment is a peristaltic pump. In some embodiments, only a single port 38 is used in which case the other port 38 (if present) is capped or otherwise closed. In still another embodiment, two ports 38 are used for filtrate extraction. The peristaltic pump 50 applies a vacuum or negative pressure to the extra-capillary space to aid in perfusion and extracting filtrate through the walls of the hollow fibers 34.

The hollow fibers 34 are dimensioned to accommodate cells within the luminal interior or intra-capillary space of the hollow fibers 34. Cells typically have diameters from about several micrometers to tens of micrometers so the hollow fibers 34 should have internal diameters larger than these sizes. The hollow fibers 34 should be dimensioned so that perfusion across the hollow fiber wall takes place without excessive clogging of the hollow fibers 34. Typical internal diameters of the hollow fibers 34 may be on the order of tens to hundreds or even thousands of microns. The hollow fibers 34 may be made of a semi-permeable material such as polysulfone, polyethersulfone, polyvinylidene fluoride, and cellulose derivatives. The permeability of the hollow fibers 34 is adjusted by the porosity of the hollow fibers 34 which is selected to allow the passage of small biomolecules (e.g., waste or product) into the filtrate located in the extra-capillary space yet allow the cells and large molecules to be retained in the retentate within the intra-capillary space. In this regard, the hollow fibers 34 may have a particular porosity or pore size or a molecular cut-off weight (MWCO) particular for the application. Typical MWCOs may include around 3 kDa and above. By matching the fiber porosity to cell characteristics, products may be accumulated, maintained, and measured on either side of the system (e.g., as filtrate or retentate). For example, in some embodiments, the final desired product may be retained in the intra-capillary space of the hollow fibers 34. This may be the case for large molecules. In other embodiments, the desired product may be small enough to pass through the semi-permeable hollow fibers 34 and accumulate in the filtrate which is removed from the hollow fiber filter 30.

In some embodiments, all of the hollow fiber filters 30 have the same MWCO or pore size. In other embodiments, the plurality of hollow fiber filters 30 may have different MWCOs or pore sizes. This allows the simultaneous creation of different fluid fractions having different compositions of biomolecules.

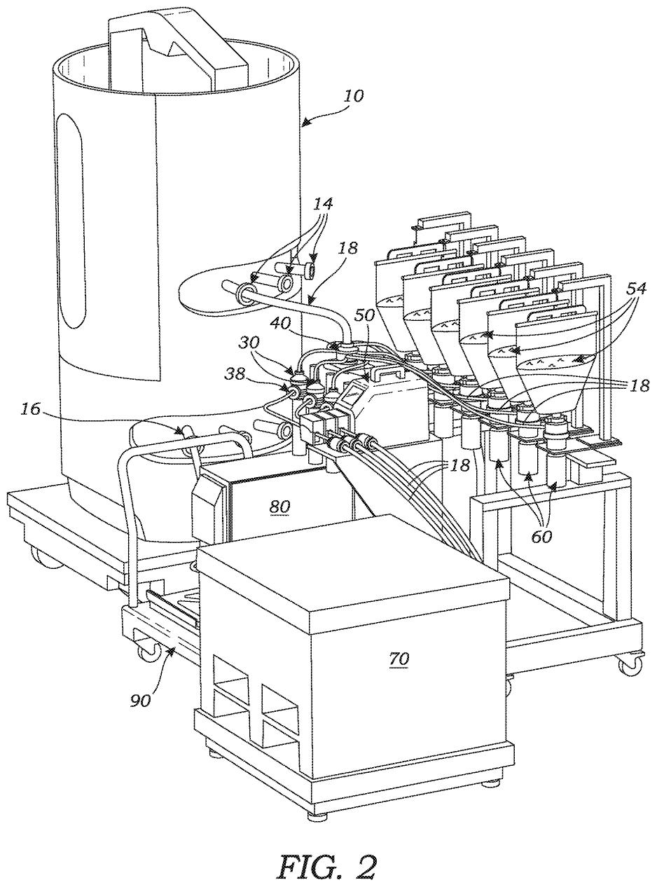

With reference to FIGS. 2-4, in another embodiment, one or more additives are provided that are combined with the cell-laden retentate prior to recirculation back to the bioreactor or vessel 10. The additives are illustrated as contained in flexible bags 54. Each bag 54 is associated with a pump 60 located at the bottom thereof that is used to pump the contents of each bag 54 to a manifold 40 that combines or mixes the additive(s) with the cell-laden retentate which then gets returned to the bioreactor or vessel 10. Also illustrated is a tote 70 that holds one or more vessel(s) or container(s) for receiving the filtrate from the hollow fiber filters 30. The filtrate may then be subject to further downstream processes like capture and purification. The pumps 60 used for the flexible bags 54 in this embodiment may also be similar to the multi-outlet pumps 12 described herein with the exception that they have a single outlet 22. The outlet 22 of each pump 60 is connected to tubing or conduit 18 that connects at the opposing end to the manifold 40 (FIGS. 2-4) via inlets 41. In this embodiment, as seen in FIG. 6, the manifold includes check valves 42 located in the manifold 40 that allow additives to flow only in a single direction into the manifold 40 and prevents backflow into the inlets 41. The additives may include reagents, cell growth media, supplements, base, acid, buffers, anti-foam agents, growth factors, and the like. These are added as needed during the cell culture process. Each pump 60 operates independently, thereby allowing the ability to fine tune the composition and quality of the cellular growth medium during operations. With reference to FIG. 1, if there are no additives being used in the system, the manifold 40 omits the inlets 41 for the additives and associated check valves 42. Note that the inlets 41 for the cells and media from the hollow fiber filters 30 are still utilized in this embodiment.

A control unit 80 is provided that controls the operation of the multi-outlet pump 12, the peristaltic (or other) pump 50, and the optional additive pumps 60. The control unit 80 may include a computer and/or one or more processors that receive data from sensors from various aspects of the cell culture system. The sensors may monitor flow rates, pressures, pH, osmolarity, temperature, conductivity, opacity or density, and the like. This allows for automatic adjustment of flow rates and pressures within the cell culture system. In addition, additives can be added as needed to maintain optimal growth conditions within bioreactor or vessel 10. The control unit 80 may optionally be accessed directly through a user interface (e.g., graphical user interface or GUI) associated with the control unit 80 or the control unit 80 may be remotely accessed using a network connection (e.g., wired or wireless connection).

The various components the form part of the perfusion system may be loaded on a cart or skid 90 or multiple such cars or skids 90 as seen in FIGS. 1-4. For example, the multi-outlet pump 12, hollow fiber filters 30, manifold 40, the peristaltic pump 50, and the optional additive pumps 60, and the control unit 80 may be mounted on or carried by the cart(s) or skid(s) 90. As seen in FIG. 2, a separate cart or skid 90 is used to hold the bags 54 and associated pumps 60. The cart or skid 90 may be wheeled so that it can be moved as needed.

The perfusion cell culture method described herein may be used with any number of different cell types. This includes mammalian as well as plant/fungi cells (e.g., yeast cells). In addition, while some embodiments contemplate a biomolecule being produced by the cells as the desired product, in other embodiments, the cells themselves may be the desired product. For example, T-cells from a patient may be grown for cell therapy applications.

To operate the perfusion cell culture system, cell-laden fluid from the bioreactor or vessel 10 is fluidically coupled to an inlet 14 of a multi-outlet pump 12. The cell-laden fluid is pumped out a plurality of outlets 22 of the multi-outlet pump 12, the plurality of outlets 22 fluidically connected via tubing or conduits 18 to separate hollow fiber filters 30. A filtrate solution is extracted from the hollow fiber filters 30 using one or more pumps 50 (e.g., peristaltic pump) fluidically coupled to an extra-capillary space within the hollow fiber filters 30. The retentate containing the cells is then returned from the hollow fiber filters 30 back to the bioreactor or vessel 10.

The retentate from the hollow fiber filters 30 may, in one embodiment, we combined or mixed together in a manifold 40 prior to reintroduction into the bioreactor or vessel 10. In some embodiments, the retentate is combined or mixed with one or more additives in the manifold 40 prior to returning to the bioreactor or vessel. The one or more additives may include a plurality of additives which are stored in respective flexible bags 54 and wherein each additive is pumped into the manifold with respective pumps 60 associated with the flexible bags 54. The one or more additives may include one or more of reagents, cell growth media, supplements, base, acid, buffers, anti-foam agents, and growth factors.

While embodiments of the present invention have been shown and described, various modifications may be made without departing from the scope of the present invention. For example, the invention has been described as having the cells contained in the intra-capillary space (i.e., within the hollow fibers 34) with the filtrate contained in the extra-capillary space of the hollow fiber filter 30. It should be appreciated that the method may be performed with the cells located in the extra-capillary space and the intra-capillary space within the hollow fibers 34 is cell-free. This would, of course, use the ports 38 as the inlet and outlet for the cell-laden fluid. The inlet 32 or outlet 36 of the hollow fiber filters 30 would then be fluidically connected to the peristaltic pump 50.

In this embodiment, a perfusion cell culture system includes a bioreactor or vessel 10 and a multi-outlet pump 12 having an inlet 20 fluidically connected to the bioreactor or vessel 10 and a plurality of pump outlets 22. The system further includes a plurality of hollow fiber filters 30 each having a plurality of hollow fibers 34 therein and defining an intra-capillary space inside the plurality of hollow fibers 34 and an extra-capillary space outside the plurality of hollow fibers 34, the plurality of hollow fiber filters 30 fluidically coupled at respective inlet ports 38 to the plurality of outlets 22 of the multi-outlet pump 12, wherein the respective inlet ports 38 of the hollow fiber filters 30 communicate with the extra-capillary space of a plurality of hollow fiber filters 30, the hollow fiber filters 30 further including respective outlet ports 38 that communicate with the extra-capillary space of the plurality of hollow fiber filters 30, and an inlet 32 and outlet 36 that communicate with the intra-capillary space of the hollow fibers 34 in the plurality of hollow fiber filters 30. The system includes a manifold 40 fluidically coupled to the bioreactor or vessel 10 and the respective outlet ports 38 of the plurality of hollow fiber filters 30. A second pump 50 is fluidically coupled to the respective outlets 36 of the plurality of hollow fiber filters 30 that communicate with the intra-capillary space of the hollow fibers 34 in the plurality of hollow fiber filters 30. The invention, therefore, should not be limited, except to the following claims, and their equivalents.

What is claimed is:

1. A perfusion cell culture method comprising:
fluidically coupling cell-laden fluid from a bioreactor or vessel into an inlet of a multi-outlet pump;
pumping the cell-laden fluid out a plurality of outlets of the multi-outlet pump, the plurality of outlets fluidically connected via tubing or conduits to separate hollow fiber filters;
extracting a filtrate solution from the hollow fiber filters using one or more pumps fluidically coupled to an extra-capillary space within the hollow fiber filters; and
returning a retentate containing the cells from the hollow fiber filters back to the bioreactor or vessel.

2. The method of claim 1, wherein the hollow fiber filters each comprise a housing containing a plurality of hollow fibers defining an interior, intra-capillary space for receiving the cell-laden fluid and the extra-capillary space being located outside of the plurality of hollow fibers and within the housing.

3. The method of claim 1, wherein each hollow fiber filter comprises hollow fibers having the substantially the same pore size or molecular weight cutoff.

4. The method of claim 1, wherein at least one of the hollow fiber filters comprises hollow fibers having a first pore size or first molecular weight cutoff and wherein at least one of the hollow fiber filters comprises hollow fibers having a second pore size or second molecular weight cutoff.

5. The method of claim 1, wherein the one or more pumps fluidically coupled to an extra-capillary space within the hollow fiber filters comprises a peristaltic pump.

6. The method of claim 1, wherein the extracted filtrate solution comprises a product enriched solution.

7. The method of claim 1, wherein the extracted filtrate solution comprises a waste enriched solution.

8. The method of claim 1, further comprising combining the retentate with one or more additives prior to returning to the bioreactor or vessel.

9. The method of claim 8, where the one or more additives are combined with the retentate in a manifold.

10. The method of claim 9, wherein the one or more additives comprise a plurality of additives and wherein each additive is pumped into the manifold with a respective pump.

11. The method of claim 8, wherein the one or more additives comprise one or more of reagents, cell growth media, supplements, base, acid, buffers, anti-foam agents, and growth factors.

12. The method of claim 1, further comprising adding cell culture media to the bioreactor or vessel.

13. The method of claim 1, wherein the number of separate hollow fiber filters comprise 2, 3, 4, 5, 6, 7, 8, 9, or 10 hollow fiber filters.

14. The method of claim 1, wherein the extracted filtrate solution is pumped to a vessel or container.

15. A perfusion cell culture system comprising:
a bioreactor or vessel;
a first multi-outlet pump having an inlet fluidically connected to the bioreactor or vessel and a plurality of outlets;
a plurality of hollow fiber filters fluidically coupled at respective inlets to the plurality of outlets of the first multi-outlet pump, wherein the respective inlets of the hollow fiber filters communicate with an intra-capillary space of a plurality of hollow fibers contained within respective hollow fiber filters, the hollow fiber filters further comprising respective outlets that communicate with the intra-capillary space of a plurality of hollow fibers contained within respective hollow fiber filters, and one or more ports that communicate with an extra-capillary space surrounding the hollow fibers of the hollow fiber filters;
a manifold fluidically coupled to the bioreactor or vessel and the respective outlets of the plurality of hollow fiber filters; and
a second pump fluidically coupled to the respective one or more ports of each of the plurality of hollow fiber filters.

16. The system of claim 15, further comprising one or more bags having additive(s) contained therein and respective additive pump(s) fluidically coupled to the manifold.

17. The system of claim 15, wherein the bioreactor or vessel is fluidically connected to the inlet of the first multi-outlet pump via tubing or conduit.

18. The system of claim 16, wherein the manifold comprises check valves disposed therein at inlet(s) configured to receive the additive(s) from the one or more bags via the respective additive pump(s).

19. The system of claim 15, wherein the second pump fluidically coupled to the respective one or more ports of each of the plurality of hollow fiber filters comprises a peristaltic pump.

20. A perfusion cell culture system comprising:
a bioreactor or vessel;
a first multi-outlet pump having an inlet fluidically connected to the bioreactor or vessel and a plurality of outlets;
a plurality of hollow fiber filters each having a plurality of hollow fibers therein and defining an intra-capillary space inside the plurality of hollow fibers and an extra-capillary space outside the plurality of hollow fibers, the plurality of hollow fiber filters fluidically coupled at respective inlet ports to the plurality of outlets of the first multi-outlet pump, wherein the respective inlet ports of the hollow fiber filters communicate with the extra-capillary space of a plurality of hollow fiber filters, the hollow fiber filters further comprising respective outlet ports that communicate with the extra-capillary space of the plurality of hollow fiber filters, and an inlet and outlet that communicate with the intra-capillary space of the hollow fibers in the plurality of hollow fiber filters;
a manifold fluidically coupled to the bioreactor or vessel and the respective outlet ports of the plurality of hollow fiber filters; and
a second pump fluidically coupled to the respective outlets of the plurality of hollow fiber filters that communicate with the intra-capillary space of the hollow fibers in the plurality of hollow fiber filters.
